(19) United States
(12) Patent Application Publication
(10) Pub. No.: US 2009/0220941 A1
(43) Pub. Date: Sep. 3, 2009

(54) COMPOSITIONS FOR- DETECTING OF INFLUENZA VIRUSES AND KITS AND METHODS USING SAME

(75) Inventors: Dorit Arad, Tel Aviv (IL); Assaf Ezra, Bat Yam (IL)

Correspondence Address: CANTOR COLBURN, LLP, 20 Church Street, 22nd Floor, Hartford, CT, 06103, US

(73) Assignee: MND DIAGNOSTIC LTD, Doar-na Hefer (IL)

(21) Appl. No.: 12/091,665

(22) PCT Filed: Oct. 25, 2006
(86) PCT No.: PCT/IL2006/001226

§ 371 Date: Dec. 9, 2008

Related U.S. Application Data
(60) Provisional application No. 60/729,752, filed on Oct. 25, 2005.

(51) Int. Cl. C12Q 1/70; C07K 2/00; C12N 9/26; C12Q 1/04

(52) U.S. Cl. 435/5; 530/300; 435/201; 435/34

(57) ABSTRACT

An isolated composition-of-matter comprising a sialic acid bound to a sialic acid binding domain of a polypeptide is provided. Uses thereof and kits comprising same are also provided.

### FIELD AND BACKGROUND OF THE INVENTION

The present invention relates to novel compositions that can be used for detecting an influenza virus.

Influenza A and B viruses are major causes of acute respiratory disease, with an estimated 30-50 million infections annually in the United States alone. Influenza A has been responsible for major epidemics, such as the “Spanish Flu” of 1919 which killed millions of people. Many viral and bacterial infections may present with symptoms similar to those of influenza.

Due to the multiple mutations and evolution that occurs in the different animal species different subtypes of viruses emerge every season. The avian influenza (H5N1) virus presents an unprecedented epizootic highly pathogenic virus that has crossed the species barrier in Asia. The virus caused human fatalities and further outbreaks indicate that other human populations may also be at risk. It has been shown that the H5N1 is acquiring a more pathogenic nature and has reach a 89% fatality rate among infants and young children, compared to the 2.5% mortality rate of the Spanish flu virus. To date a limited, non-sustained human-to-human transmission of H5N1 has been reported. Evidence is consistent with bird-to-human, possibly environment-to-human transmission of H5N1. Once the H5N1 strain will acquire an efficient human-to-human transmission the risk of a pandemic will increase dramatically.

Laboratory tests for the identification of viruses in clinical material are widely used, and a variety of different detection methodology is available. The textbook, “Laboratory Diagnosis of Viral Infections”, Marcel Dekker 1992, Ed E. H. Lennette generally discusses methods which are used for a wide range of viruses, including influenza.

A number of tests are available for the diagnosis of influenza A and B. The traditional method of identifying influenza viruses has been the use of cell culture, which is highly sensitive and specific. Unfortunately, the time required for culture, isolation and identification of influenza virus can range between 2 and 10 days, thus making it virtually useless in guiding the physician to an appropriate therapy. Since influenza virus infection is normally self-limiting, diagnosis must be rapid if therapy is to be effective. In other words, such cell culture methods are normally only of value in providing retrospective epidemiological information.

The majority of influenza detection methods and the methods that differentiate between influenza strains or subtypes detect the presence of the influenza genetic material through RT-PCR or PCR [Hassibi et. al., Biophys Chem. 2005 Aug. 1;116(3):175-85. Hum Mutat. 2006 Jul.;27(7):644-53; Claas E C, et al., Lancet. 1998; 351,p. 472-477]. These methods employ different measures to detect the result of the genetic amplification reaction. RT-PCR and PCR reactions are complicated and thus require expert lab work and might take up to 8 hours.

Some detection methods are based on monoclonal antibodies specific for either type A or B nucleoprotein [U.S. Pat. No. 5,316,910] or for either H1N1 or H2N2 subtypes haemagglutinin [U.S. Pat. No. 5,589,174]. An immuno based assay however is labor-intensive, and requires considerable technical expertise, with the results often being difficult to interpret.

Recently, a few rapid direct tests have become available, which are specific for influenza A. Thus, a monoclonal immunofluorescence assay (IFA) has been reported (Spada, B. et al, J. Virol. Methods, 1991 33 305) and at least one enzyme immunoassay (EIA) is available (Ryan-Poirier, K. A. et al, J. Clin. Microbiol., 1992 30 1072). A number of comparisons of these rapid detection methods for influenza A have been reported; see for example Leonardi, G. P. et al, J. Clin. Microbiol., 1994 32 70, who recommended that direct specimen testing be used together with culture isolation, so as to permit both identification of the virus, in time to institute therapy and infection control measures, and to monitor the antigenic constitution of influenza strains prevalent in the community for epidemiological purposes. The IFA method is reported to be labor-intensive, and requires considerable technical expertise, with the results often being difficult to interpret. On the other hand, the ETA method (Directigen FLU-A; Becton Dickinson Microbiology Systems) gave a high level of false-positive results, and it has been recommended that this assay should be used in laboratories only in addition to or as a substitute for direct immunofluorescence tests (Waner, J. L. et al, J. Clin. Microbiol., 1991 29 479).

Influenza virions contain two glycoproteins on their surface, hemagglutinin (HA) and neuraminidase (NA), which recognize sialic acid on host cell glycoconjugates. HA binds to cell surface sialyloligosaccharides and then mediates the entry of the virus into the cell, while NA prevents the aggregation of progeny virions by removing sialic acid on the oligosaccharides of newly synthesized HA and NA polypeptides. The glycosidic linkage bonding sialic acid (Neu5Ac) with the penultimate saccharide is the substrate of the neuraminidase activity of the influenza virion. The neuraminidase hydrolyzes the linkage, thereby cleaving Neu5Ac from the penultimate saccharide. NA also facilitates the elution of progeny virions from infected cells by removing sialic acid from host cell glycoconjugates [Zambion, J. antimicrobial chemotherapy, 1999, 44, Topic B, 3-9].

The crucial role HA and NA play in the infection of the influenza virus have placed them at the center of attention in influenza research and numerous studies have examined the specificities and preference of HA and NA of the different strains to different substrates.

The HA receptor specificity of influenza viruses differs according to the host species of origin. That is, HA preferentially recognizes the sialic acid-galactose linkages expressed on cells of the host from which the virus was isolated. For example, duck intestinal epithelial cells express primarily N-acetylneuraminic acid (NeuAc) bound to galactose through an α2,3 linkage (NeuAcα2-3Gal), which is preferentially recognized by duck influenza virus HA. Likewise, human virus HA preferentially binds NeuAcα2-6Gal, the primary linkage of sialic acid expressed on human tracheal epithelial cells. Similarly, the NA specificity for NeuAcα2-3Gal and NeuAcα2-6Gal depends on the viral isolate examined.

U.S. Pat. No. 5,252,458 and U.S. Pat. No. 7,081,352 both teach enzymatic based detection methods for the identification of the influenza virus. The enzymatic based assays taught therein detect the presence of a virus through the reaction of a viral neuraminidase with a chromogenic substrate. The chromogenic substrate decomposes to produce light which can then be detected. The chemiluminescent substrate materials include enzymatically triggerable 1,2-dioxetane derivatives of 4-alkoxy-N-acetylneuraminic acid and 4,7-dialkoxy-N-acetyineuraminic acid and other N-neurameric derivatives. U.S. Pat. No. 5,663,055 teaches chromogenic derivatives of N-acetylneuraminic acid that exhibit different reactivity with the different types of influenza neuraminidases, thus enabling one to discern the specific type of influenza infection. Thus, influenza type A and B viruses can be distinguished from parainfluenza type 1, 2, 3, and 4, and mumps using the 4,7-dialkoxy-N-acetylneuraminic acid. This substrate however, cannot distinguish between different strains of influenza which also include the HA subtyping.

Due to multiple mutations and evolution, the influenza virus develops different subtypes every season. Since there is a global fear that one mutated type may become particularly violent, there is an urgent need to rapidly detect and isolate the violent types of influenza virus both in humans and in birds, in order to monitor and minimize the spread of the pandemic threatened to occur by such viruses.

There is thus a widely recognized need for, and it would be highly advantageous to have, methods and substrates that may be used for the rapid detection different strains of influenza, specifically ones that can become very violent and lethal for people such as H5N1.

### SUMMARY OF THE INVENTION

According to one aspect of the present invention there is provided an isolated composition-of-matter comprising a sialic acid bound to a sialic acid binding domain of a polypeptide.

According to another aspect of the present invention there is provided a composition being of the general formula:

X—Y-Z

wherein:

Y comprises a substrate of a neuraminidase, cleavage of X—Y-Z by the neuraminidase forming cleavage products X—Y′ and Y″-Z wherein Y′ is a first cleavage product of Y and Y″ is a second cleavage product of Y;

X comprises a detectable moiety; and

Z comprises a separating moiety capable of binding to a separate phase of a two phase separating system;

wherein the X—Y-Z does not form a contiguous portion of a natural substrate of the viral neuraminidase.

According to yet another aspect of the present invention there is provided a method for detecting at least one pathogen in a sample, the method comprising:

(a) contacting the sample with the compositions of the present invention under conditions allowing cleavage of the substrate; and

(b) monitoring cleavage of the substrate, wherein the cleavage of the substrate is indicative of the presence of the at least one pathogen in the sample.

According to yet an additional aspect of the present invention there is provided method of detecting a potentially human pathogenic avian influenza virus in a bird, the method comprising

(a) contacting an avian sample with a neuraminidase substrate, the neuraminidase substrate comprising an α2,6 linkage; and

(b) monitoring cleavage of the substrate, wherein the cleavage of the substrate is indicative of a potentially human pathogenic avian influenza virus.

According to still an additional aspect of the present invention there is provided a method of detecting an avian influenza virus in a human, the method comprising (a) contacting a human sample with a neuraminidase substrate comprising a NeuGcα2,3 linkage; and (b)monitoring cleavage of the substrate, wherein the cleavage of the substrate is indicative of an avian influenza virus in the human.

According to a further aspect of the present invention there is provided a diagnostic kit for detection of at least one influenza virus in a sample, the kit comprising a plurality of compositions of the present invention, and reagents for detecting cleavage of the substrate.

According to yet a further aspect of the present invention there is provided diagnostic kit comprising a packaging material and a plurality of compositions for detecting presence of a plurality of influenza viruses in a sample, wherein each of the compositions is of a general formula,

X—Y-Z

wherein:

Y comprises a substrate of a viral neuraminidase, cleavage of X—Y-Z by the viral neuraminidase forming cleavage products X—Y′ and Y″-Z wherein Y′ is a first cleavage product of Y and Y″ is a second cleavage product of Y;

X comprises a detectable moiety; and

Z comprises a separating moiety capable of binding to a separate phase of a two phase separating system;

wherein the X—Y-Z does not form a contiguous portion of a natural substrate of the neuraminidase,

wherein each of the X is distinctively detectable and whereas the packaging material comprises a label or package insert indicating that the kit is for detection of plurality of influenza viruses in a sample.

According to further features in preferred embodiments of the invention described below, the sialic acid is attached to at least one carbohydrate via a linker.

According to still further features in the described preferred embodiments, the sialic acid is attached to two carbohydrates wherein a first carbohydrate of the two carbohydrates is attached to a second carbohydrate of the two carbohydrates via a second linker.

According to still further features in the described preferred embodiments, the second linker is selected from the group consisting of an α1-4 linker, an α1-3 linker, an α2-3 linker and an α1-6 linker.

According to still further features in the described preferred embodiments, the composition is hydrolizable by neuraminidase.

According to still further features in the described preferred embodiments, the carbohydrate is selected from the group consisting of galactose, N-acetylgalactosamine, glucose, mannose, glucoseamine galactoseamine, rhamnose, fucose, inositol, scyllo-inositol, fructose, xylulose, ribulose, ribose, aloes, altrose, arabinose, xylose, gulose, idose, lyxose, talose, neuraminic acid, glucoronic acid glucaric acid, gluconic acid, GalNAc and GlcNAc.

According to still further features in the described preferred embodiments, the linker is selected from the group consisting of an α2,3 linker, an α2,6 linker and an α2,8 linker.

According to still further features in the described preferred embodiments, the sialic acid is naturally occurring.

According to still further features in the described preferred embodiments, the sialic acid is synthetic.

According to still further features in the described preferred embodiments, the polypeptide is a hemagglutinin polypeptide.

According to still further features in the described preferred embodiments, the sialic acid binding domain is comprised in a hemagglutinin polypeptide.

According to still further features in the described preferred embodiments, the hemagglutinin polypeptide is truncated.

According to still further features in the described preferred embodiments, the hemagglutinin polypeptide is selected from the group consisting of an H1 polypeptide, an H2 polypeptide, an H3 polypeptide, an H4 polypeptide, an H5 polypeptide, an H6 polypeptide an H7 polypeptide, an H8 polypeptide, an H9 polypeptide, an H10 polypeptide, an H11 polypeptide, an H12 polypeptide, an H13 polypeptide, an H14 polypeptide, an H15 polypeptide and an H16 polypeptide.

According to still further features in the described preferred embodiments, the composition further comprises at least one detectable moiety.

According to still further features in the described preferred embodiments, the sialic acid is attached to at least one carbohydrate via a linker.

According to still further features in the described preferred embodiments, the detectable moiety is attached to the sialic acid via a linker.

According to still further features in the described preferred embodiments, the at least one detectable moiety is a pre-enzyme, and whereas cleavage of the linkage activates the pre-enzyme.

According to still further features in the described preferred embodiments, the at least one detectable moiety is a pre-substrate and whereas cleavage of the linkage releases the pre-substrate to form an active detectable substrate of an enzyme.

According to still further features in the described preferred embodiments, the enzyme is β galactosidase.

According to still further features in the described preferred embodiments, the at least one detectable moiety is a FRET pair, and whereas cleavage of the linkage generates a signal from the FRET pair.

According to still further features in the described preferred embodiments, the at least one detectable moiety is a Quantum dot, and whereas cleavage of the linkage generates a signal from the Quantum dot.

According to still further features in the described preferred embodiments, the at least one detectable moiety comprises a labeling agent selected from the group consisting of an enzyme, a fluorophore, a chromophore, a protein, a peptide, a pre-enzyme, a chemiluminescent substance, a pre-substrate, Quantum Dot and a radioisotope.

According to still further features in the described preferred embodiments, the composition of matter further comprises a separating moiety.

According to still further features in the described preferred embodiments, the composition-of-matter is of the general formula:

X—Y-Z

wherein:

Y comprises a substrate of a neuraminidase, cleavage of X—Y-Z by neuraminidase forming cleavage products X—Y′ and Y″-Z wherein Y′ is a first cleavage product of Y and Y″ is a second cleavage product of Y;

X comprises a detectable moiety; and

Z comprises the separating moiety capable of binding to a separate phase of a two phase separating system;

wherein the X—Y-Z does not form a contiguous portion of a natural substrate of the viral neuraminidase.

According to still further features in the described preferred embodiments, the detectable moiety comprises a labeling agent selected from the group consisting of an enzyme, a fluorophore, a chromophore, a FRET pair, a protein, a peptide, a pre-enzyme, a chemiluminescent substance, a pre-substrate, Quantum Dot and a radioisotope.

According to still further features in the described preferred embodiments, the separating moiety is selected from the group consisting of an immunological binding agent, a magnetic binding moiety, a peptide binding moiety, an affinity binding moiety and a nucleic acid moiety.

According to still further features in the described preferred embodiments, the separating moiety further comprises a detectable moiety.

According to still further features in the described preferred embodiments, the pathogen is an influenza virus.

According to still further features in the described preferred embodiments, the sample is a mammalian sample.

According to still further features in the described preferred embodiments, the mammalian sample is a human sample.

According to still further features in the described preferred embodiments, the sample is an avian sample.

According to still further features in the described preferred embodiments, when the substrate comprises an α2,6 linkage, the cleavage of the substrate is indicative of a potentially human pathogenic avian influenza virus.

According to still further features in the described preferred embodiments, when the substrate comprises an NeuGcα2,3 linkage, the cleavage of the substrate is indicative of avian influenza virus.

According to still further features in the described preferred embodiments, the sample is selected from the group consisting of mucus, saliva, throat wash, nasal wash, spinal fluid, sputum, urine, semen, sweat, feces, plasma, blood, broncheoalveolar fluid, vaginal fluid, tear fluid and tissue biopsy.

According to still further features in the described preferred embodiments, the sample is selected from the group consisting of mucus, saliva, throat wash, nasal wash, spinal fluid, sputum, plasma, blood, broncheoalveolar fluid, tear fluid and tissue biopsy.

According to still further features in the described preferred embodiments, the monitoring is effected using a homogeneous assay.

According to still further features in the described preferred embodiments, the monitoring is effected using a heterogeneous assay.

According to still further features in the described preferred embodiments, the substrate of the viral neuraminidase comprises a sialic acid bound to a sialic acid binding domain of a polypeptide.

According to still further features in the described preferred embodiments, the diagnostic kit further comprises reagents for detecting cleavage of the substrate.

According to still further features in the described preferred embodiments, the each of the plurality of compositions is a substrate for a different neuraminidase.

According to still further features in the described preferred embodiments, the plurality of compositions are attached to a single solid support.

According to still further features in the described preferred embodiments, the distinctive detection is effected by an addressable location on the single solid support.

According to still further features in the described preferred embodiments, the distinctive detection is effected by different detectable moieties.

According to still further features in the described preferred embodiments, the solid support is configured as a bead.

According to still further features in the described preferred embodiments, the bead is selected from the group consisting of a colored bead, a magnetic bead, a tagged bead and a fluorescent bead.

According to still further features in the described preferred embodiments, the diagnostic kit further comprises a substrate for the detection of at least one more virus other than the influenza virus selected from the group consisting of Corona Viruses, SARS, HMPV (Human Meta pneumo virus), Adeno virus, RSV (Respiratory Syncytial Virus) and Rhino virus.

The present invention successfully addresses the shortcomings of the presently known configurations by providing methods and compositions of matter for the rapid detection of a particular strain of influenza virus.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic representation of the structure of one embodiment of the substrate of the present invention.

FIG. 2 is a schematic representation of the basic operation of one embodiment of the separation system of the present invention.

FIG. 3 is a schematic representation of the simultaneous detection of three substrate molecules, wherein two of them undergo cleavage.

FIG. 4 is a schematic representation of a dynamic separation system according to one embodiment of the present invention.

FIG. 5 is a schematic representation of exemplary sialic acids that may be used according to the present invention.

FIG. 6 is a schematic representation of a neuraminidase substrate for the detection of influenza virus.

FIG. 7 is a schematic representation depicting the specificity of a human influenza virus and an avian influenza virus for a human or avian cell respectively.

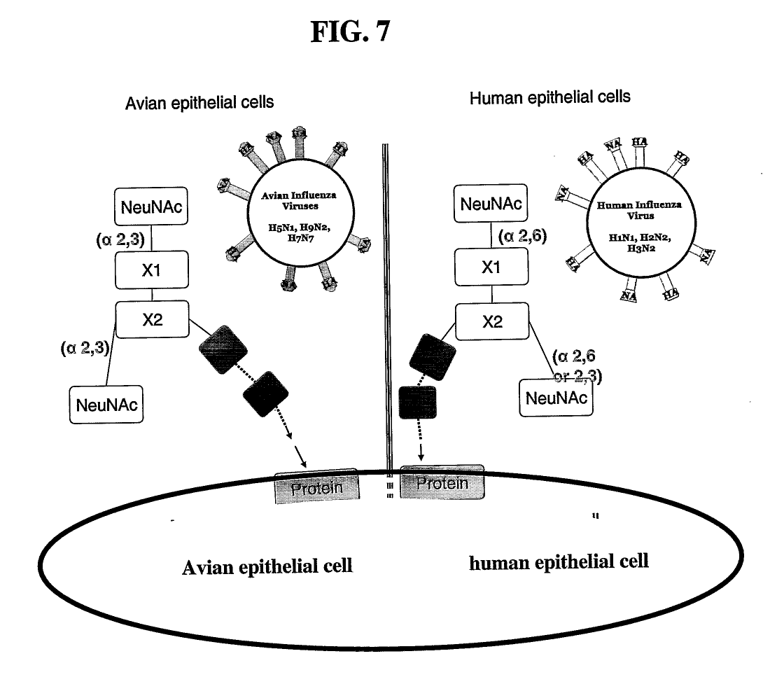

FIGS. 8A-C is a photograph of an exemplary kit for testing the pathogenicity of avian flu virus. FIG. 8A is a photograph of the kit following contact with a sample not comprising influenza virus. FIG. 8B is a photograph of the kit following contact with a sample comprising human non-pathogenic avian influenza. FIG. 8C is a photograph of the kit following contact with a sample comprising human pathogenic avian influenza.

FIG. 9 is a block diagram illustrating the process of influenza virus detection.

FIG. 10 is a schematic diagram illustrating detection of substrate cleavage using a pre-substrate.

FIG. 11 is a schematic diagram for the simultaneous detection of different types of influenza with colored beads.

### DESCRIPTION OF THE PREFERRED EMBODIMENTS

The present invention is of a novel substrate for neuraminidase. Specifically, the present invention can be used to detect viruses which express neuraminidase and differentiate between strains thereof.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Diagnosis of viral infection, such as infection by influenza virus, may be carried out by detecting the presence of unique moieties characteristic of the virus. Virus particles typically carry distinctive antigenic components on the exterior of the virion which may be detected by specific ligand-anti-ligand interactions (e.g. ligand binding moieties, receptors and antibodies), in particular by the use of an antibody specific for a viral epitope. Such interactions rely on the law of mass action, and for this reason may have limited sensitivity. Many virus particles additionally carry specific enzymatic activities on the virion particle. Influenza virus is an example of such a virus, endowed with a virus-specific neuraminidase activity as an integral part of the virion which is exposed to the environment. Its substrate is the glycosidic linkage bonding sialic acid (Neu5Ac) with the penultimate saccharide of host cell surface glycoconjugates. Neuraminidase hydrolyzes the linkage, thereby cleaving Neu5Ac from the penultimate saccharide.

Utilization of the enzymatic activity in such cases offers the potential for increasing the sensitivity of a detection method.

Influenza virions also comprise hemagglutinin (HA) on their surface which, like neuraminidase, also recognizes terminal sialic acids on host cell glucoconjugates. These terminal sialic acids serves as ligands to the HA sialic acid receptor domain. The HA receptor and NA specificity of influenza viruses differs according to the host species of origin. That is, HA and NA preferentially recognizes the sialic acid-galactose linkages expressed on cells of the host from which the virus was isolated. The HA-sialic acid complex interacts with the NA to mediate the cleavage of the sialic acid glycosidic linkage on the penultimate saccharide of the glycoconjugates. Many variants of HA (1-16) and NA (1-9) exist, and it is evident that not every combination of NA and HA can form an infective virus. The close interactions between NA and HA towards the sialic acid requires compatibility between these two cell surface particles in order to achieve efficient cleavage.

Whilst conceiving the present invention, the present inventors have conceptualized a novel method for differentiating between influenza virus strains according to their HA-sialic acid gycoconjugate-NA complex specificity. Accordingly, the present inventors envisage a novel neuraminidases substrate molecule comprising sialic acid bound to an HA polypeptide, or portion thereof. Incorporation of the HA polypeptide in the substrate molecule allows for a greater level of differentiation between influenza viruses since it serves to present the sialic acid on the cell surface in a particular fashion thereby enabling the cleavage of this complex by only certain neuraminidases.

Furthermore, the present inventors provide substrates capable of differentiating between human pathogenic avian influenza viruses and human non-pathogenic avian influenza viruses. Such substrates comprise sialic acid bound to a sugar moiety, wherein the linkage between the two provides specificity for either a pathogenic or non-pathogenic avian influenza virus. Specifically, cleavage of sialic acid from a 2,6 linked sugar as well as a 2,3 linked sugar (or 2,8 linked sugar) will indicate that the avian virus is potentially pathogenic to humans.

Thus, according to one aspect of the present invention, there is provided an isolated composition-of-matter comprising a sialic acid bound to a sialic acid binding domain of a polypeptide.

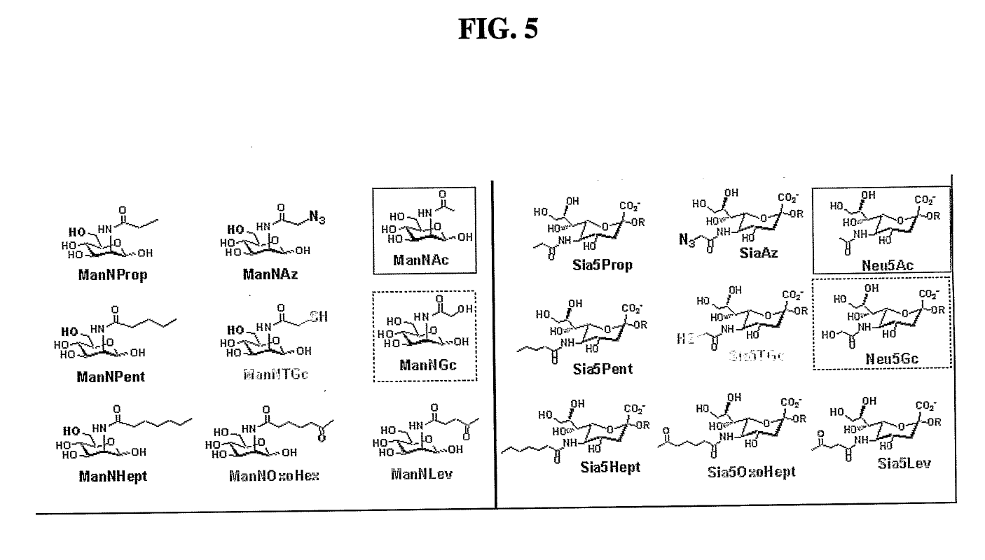

As used herein, the phrase “sialic acid” refers to any member of a family of SIA derivatives. The most common member of the sialic acid family is N-acetyl-neuraminic acid (2-keto-5-acetamindo-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al. (1990) J. Biol. Chem. 265: 21811-21819. Also included are 9-substituted sialic acids such as a 9-O-C1-C6 acyl-Neu5Ac like 9-O-lactyl-Neu5Ac or 9-O-acetyl-Neu5Ac, 9deoxy-9-fluoro-Neu5Ac and 9-azido-9-deoxy-Neu5Ac. For review of the sialic acid family, see, e.g., Varki (1992) Glycobiology 2: 25-40; Sialic Acids: Chemistry, Metabolism and Function, R. Schauer, Ed. (Springer-Verlag, New York (1992). The sialic acids may be naturally occurring or synthetic (i.e. analogues). Exemplary sialic acids and analogues thereof (including ManAc analogues) are illustrated in FIG. 5.

Methods of synthesizing sialic acids are well known in the art—see for example “Carbohydrates in Drug Design” (1999, 64, No. 7, 847), “Glycopeptides and Related Compounds. Synthesis, Analysis, and Applications” (1999, 64, No. 9, 1092), “Techniques in Glycobiology” (1999, 64, No. 10, 1215) and Glycochemistry. Principles, Synthesis, and Applications (Wang, P. G., and Bertozzi, C. R., eds., Marcel Dekker, New York-Basel, 2001.

As used herein, the term “bound” refers to any type of bonding including but not limited to covalent bonding, hydrogen bonding, electrostatic binding and receptor substrate affinity bonding.

According to this aspect of the present invention, the sialic acid in the substrate molecule is bound to a sialic acid binding domain of a polypeptide.

As used herein, the phrase “sialic acid binding domain of a polypeptide” refers to any peptide sequence (i.e., naturally occurring or synthetic) that is capable of binding sialic acid and presenting it such that it is capable of being catalytically processed/hydrolyzed by neuraminidase.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, Tic, naphtylalanine (Nal), phenylisoserine, threoninol, ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (e.g., synthetic, Table 2) which can be used with the present invention.

TABLE 1

| | Three-Letter | One-letter |
|---|---|---|
| Amino Acid | Abbreviation | Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-metyhylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The sialic binding domain may be a short peptide sequence (e.g. less than 10 amino acids) that just comprises the sialic acid binding domain, or be comprised in a polypeptide that comprises a sialic acid binding domain as well as other domains e.g. a full length protein or a truncated protein (e.g., 10-50 amino acids). Exemplary short peptide sequences that are capable of binding sialic acids are known in the art, see for example Heerze et al Glycobiology vol 5 no 4 pp. 427-433, 1995.

Examples of polypeptides that are capable of binding sialic acids (i.e. proteins that comprise sialic acid binding domains) are set forth in Table 3 hereinbelow.

TABLE 3
Vertebrate
C-type: Selectins
I-type: Siglecs
Unclassified: Complement factor H, laminin
Anthrapod
Crab lectins: Limulin (American horseshoe crab, Limulus polyphemus)
Lobster and prawn lectins: 1-agglutinin (lobster, Homarus americanus)
Scorpion lectins: Whip scorpion lectin (Masticoproctus giganteus)
Other insect lectins: Allo A-II (beetle lectin, Allomyrina dichotoma)
Mollusc
Slug and snail lectins: Limax flavus agglutinin (LFA, slug, Limax flavus)
Mussel and oyster lectins: Pacific oyster lectin (Crassostrea gigas)
Protozoal
Parasite lectins: Merozoite erythrocyte-binding antigen (Plasmodium falciparum)
Plant
SN agglutinin (elderberry bark lectin, Sambucus nigra), TJ agglutinin (Tricosanthes japonicum),
MA agglutinin (Maackia amurensis), wheat-germ agglutinin (Triticum vulgaris)
(Tricosanthes japonicum), MA agglutinin (Maackia amurensis), wheat-germ agglutinin
(Triticum vulgaris)
Bacterial
Bacterial adhesins: S-adhesin (Escherichia coli K99), adhesin I and adhesin II (Helicobacter pylori)
Bacterial toxins: Cholera toxin (Vibrio cholerae), tetanus toxin (Clostridium tetani),
botulinum toxin (Clostridium botulinum), pertussis toxin (Bordetella pertussis)
Mycoplasma lectins: Mycoplasma pneumoniae hemagglutinin
Viral
Hemagglutinins: Influenza A and B viruses, Primate polyomaviruses, Rotaviruses
Hemagglutinin-neuraminidases: New Castle disease virus, Sendai virus, fowl plague virus,
parainfluenza
Hemagglutinin esterases: Influenza C viruses, human and bovine coronaviruses

According to a preferred embodiment of this aspect of the present invention the present invention, the sialic acid binding domain is a hemagluttinin polypeptide, or part thereof e.g. one of the 16 influenza hemagluttinin variants (H1, H2, H3 H4, H5, H6, H7, H8, H9, H10, H11, H12, H13, H14, H15 or H16. Preferably the amino acid sequence of the hemagluttinin polypeptide of the present invention is at least 80% homologous to the naturally occurring sequence.

Short peptides that comprise sialic acid binding sites can be synthesized using solid phase peptide synthesis procedures that are well known in the art and further described by John Morrow Stewart and Janis Dillaha Young, [Solid Phase Peptide Syntheses (2nd Ed., Pierce Chemical Company, 1984)]. Synthetic peptides can be purified by preparative high performance liquid chromatography [Creighton T. (1983) Proteins, structures and molecular principles. W H Freeman and Co. N.Y.] and the composition of which can be confirmed by amino acid sequencing.

In cases where longer peptide are desired e.g. full length proteins or truncated proteins, they can be generated using recombinant techniques such as described by Bitter et al., (1987) Methods in Enzymol. 153:516-544, Studier et al., (1990) Methods in Enzymol. 185:60-89, Brisson et al., (1984) Nature 310:511-514, Takamatsu et al., (1987) EMBO J. 6:307-311, Coruzzi et al., (1984) EMBO J. 3:1671-1680 and Brogli et al., (1984) Science 224:838-843, Gurley et al., (1986) Mol. Cell. Biol. 6:559-565 and Weissbach & Weissbach, 1988, Methods for Plant Molecular Biology, Academic Press, N.Y., Section VIII, pp 421-463.

According to a particularly preferred embodiment, the sialic acid of the composition of the present invention is attached to at least one carbohydrate via a linker in such a way that it is a substrate for neuraminidase (i.e. hydrolyzed by neuraminidase).

As used herein, the term “neuraminidase” refers to the enzyme also known as sialidase, acylneuraminyl hydrolase as set forth in EC 3.2.1.18.

Exemplary carbohydrates according to this embodiment of this aspect of the present invention include, but are not limited to galactose, N-acetylgalactosamine, glucose, mannose, glucoseamine galactoseamine, rhamnose, fucose, inositol, scyllo-inositol, fructose, xylulose, ribulose, ribose, aloes, altrose, arabinose, xylose, gulose, idose, lyxose, talose, neuraminic acid, glucoronic acid glucaric acid, gluconic acid GalNAc and GlcNAc.

As used herein, the term “linker” refers to a bond, preferably a covalent bond. The link between the carbohydrate and sialic acid may be an α2, 3 link, an α2, 6 link or an α2, 8 link.

The sialic acid may be naturally bound to such a carbohydrate moiety. Alternatively, the carbohydrate may be bound to the sialic acid using chemical procedures which are well known in the art.

The substrate composition of the present invention may comprise a second carbohydrate moiety attached to the first carbohydrate moiety, further mimicking the natural substrate of neuraminidase. Any link is envisaged between the two carbohydrate moieties including for example an α1-4 link, an α1-3 link, an α2-3 link and an α1-6 link.

Since the compositions of the present invention are intended for diagnostic, they preferably comprise a detectable moiety.

As used herein a detectable moiety (also referred to herein as a signaling moiety) refers to a molecule or molecules which can be directly or indirectly detected (visualized, counted etc.). Exemplary detectable moieties include, but are not limited to an enzyme, a pre-enzyme, a pre-substrate, a fluorophore, a chromophore, a protein (e.g., an epitope tag), a peptide, a quantum dot, a chemiluminescent substance, a FRET pair and a radioisotope.

According to one scenario, the detectable moiety is bound to the composition of the present invention via a linker so that the link between the detectable moiety and the sialic acid itself is the cleavage site for the neuraminidase.

According to another scenario, the detectable moiety is bound to the composition of the present invention which already comprises a link which acts as a cleavage site for neuraminidase (e.g. comprises a carbohydrate linked by an α2, 3 or α2, 6 link). In this case the detectable moiety is not bound to the sialic acid moiety via a potential substrate link.

Preferably, the detectable moiety provides a signal upon cleavage of the linker.

An example of such a detectable moiety is a pre-enzyme. Accordingly upon substrate cleavage the enzyme can be activated and detected (via the detection of a catalytic activity of same). An example of such a pre-enzyme is pro-Thrombin (factor II) or other enzymes in this cascade.

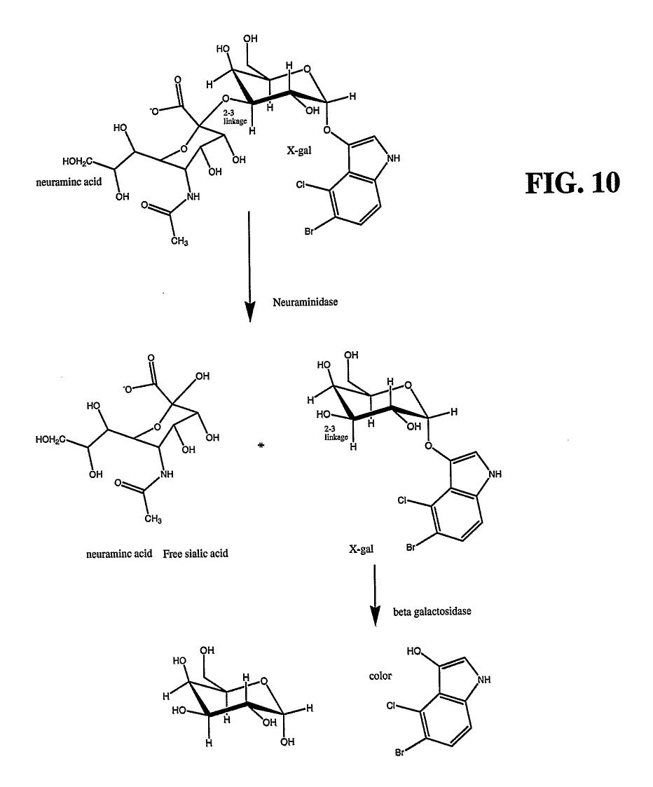

Another example of such a detectable moiety is a pre-substrate. Accordingly, upon substrate cleavage, the pre-substrate turns it into an active substrate for a second enzyme. For example the composition of matter of the present invention may comprise NeuAc(2,3)-Xgal. The X-gal is combined with the sialic acid in such a way that it cannot be cleaved by β-galactosidase. Cleavage of the pre-substrate releases free Xgal which becomes a substrate for the β-galactosidase enzyme. Subsequent cleavage of X gal by β-galactosidase produces a detectable color. Pre-substrates are further discussed in Example 8 and illustrated in FIG. 10.

Yet another example of such a detectable moiety is a quantum dot. Accordingly upon substrate cleavage, a signal is released from the quantum dot.

A further example of such a detectable moiety is a FRET pair wherein cleavage of the linkage releases the quencher and a FRET signal is generated. For the purposes of the invention, a “quencher moiety” is any substance that is capable of reducing or eliminating the signal emitted by the signaling moiety. For example, the quencher moiety may act by absorption of the signal emitted by the signaling moiety or by an energy transfer mechanism. The distance between the signaling moiety and the quencher moiety is such that presence of the quencher moiety substantially reduces or eliminates the signal emitted from the signaling moiety unless the substrate is cleaved at a position resulting in separation of the signaling and quencher moieties.

Typically one member of the FRET pair is placed on one end of the cleavage sequence, and the other member is placed on the other end of the cleavage sequence. It will be appreciated, however, that if needed (pending on the configuration of detetion) all the moieties may be placed on one end of the cleavage sequence.

An exemplary method for preparing a substrate molecule of the present invention comprising a FRET pair is described hereinbelow.

First, the polypeptide sialic acid binding site (e.g. HA) is labeled with a quencher by using NHS ester reactive dyes. Labeling may be accomplished using a reactive succinimdyl-ester (NHS). A covalent amide linkage may be formed between the activated dye and available amino groups (i.e. lysine or N terminal in proteins). One example for labeling using NHS dyes is Fluoro•Spin 331 Protein Labeling & Purification Kit, by emp Biotech GmbH. This dye is designed for the labeling of proteins with molecular weights greater than 25 kD, using a reactive succinimidyl-ester of N-methanthranilic acid (MANT). The conjugates result from the formation of a stable covalent amide linkage. The protein-dye conjugates have fluorescence-excitation and fluorescence-emission maxima at around 331 nm and 426 nm, respectively. The same principle can be applied to label HA with dabcyl-N-hydroxysuccinimide ester (dabcyl-NHS; available also from Molecular Probes, Inc, Eugene, Oreg., USA,. D-2245) for example. The result will be HA-dabcyl conjugate.

Next, the sialic acid moiety attached to a carbohydrate sequence is attached to a fluorphore (EDANS) down stream to the sialic acid on one of the carbohydrates in the sequence. The final stage is to connect the labeled sialic acid and HA via ligand-receptor interactions. In this way the fluorophore (EDANS) is placed in close proximity to the quencher (DABCYL) and FRET is made possible. Upon cleavage with NA the fluorophore is released and can be detected.

In one embodiment, the signaling moiety and quencher moiety are separated by no more than 3 or 5 amino acid residues. In another embodiment, the signaling moiety and quencher moiety are separated by no more than 10 amino acid residues. In yet another embodiment, the signaling moiety and quencher moiety are separated by no more than 15 amino acid residues. In yet another embodiment, the signaling moiety and quencher moiety are separated by no more than 20 amino acid residues. Other moieties which are used as means for detection such as further described hereinbelow may be conjugated according to these guidelines. Also, it will be appreciated that any of the detection means (e.g., moieties) may be conjugated directly or indirectly to the substrate either sequentially or by amino acid modification to any one of the amino acids of the peptide cleavage sequence itself.

Fluorescent and colorimetric assays are known to those skilled in the art. See, for example: Biochemistry, Allinger, Wang Q. M. et al., “A continuous calorimetric assay for rhinovirus- 14 3C protease using peptide p-nitroanilides as substrates” Anal. Biochem. Vol. 252, pp. 238-45 (1997), and Basak S. et al. “In vitro elucidation of substrate specificity and bioassay of proprotein convertase 4 using intramolecularly quenched fluorogenic peptides” Biochem. J. Vol. 380, pp. 505-14 (2004).

In another embodiment of the present invention, the signaling moiety (i.e. detectable moiety) is a chemiluminescent signaling moiety. The chemiluminescent signaling moiety is attached to one side of the neuraminidase cleavage region of the substrate and a fluorescent accepting quencher moiety is attached at the other side of the cleavage region. U.S. Pat. No. 6,243,980, the contents of which are incorporated by reference, describes such a detection system, involving the use of a chemiluminescent 1,2-dioxetane compound as the signaling moiety. If the viral neuraminidase is not present in the sample, cleavage of the substrate does not occur. The energy from the 1,2-dioxetane decomposition is transferred to the fluorescent accepting moiety and released at a wavelength distinct from the emission spectrum of the 1,2-dioxetane. If the substrate is cleaved, the fluorescent accepting moiety is separated from the 1,2-dioxetane and a chemiluminescent emission from the dioxetane compound is observed.

In another embodiment, the signaling moiety is a fluorescent compound and the quencher moiety is a fluorescent compound having an excitation spectrum that overlaps the emission spectrum of the signaling moiety. Here, the two moieties are separated apart at a distance consistent with fluorescent resonance energy transfer so that the fluorescent moiety is capable of acting as a resonance energy donor.

In another embodiment, a quenching group, such as a non-fluorescent absorbing dye is used in place of the fluorescent accepting quenching moiety. Suitable quenching groups are described in U.S. Pat. No. 6,243,980, the contents of which are incorporated by reference.

According to another embodiment of this aspect of the present invention, the substrate composition of the present invention comprises a separating moiety. Such substrate configurations are preferably used in heterogeneous detection assays which are further described hereinbelow.

As used herein the phrase “separating moiety” is any moiety which allows separation of uncleaved and cleaved products. Exemplary separating moieties include, but are not limited to immunological binding agent, a magnetic binding moiety, a peptide binding moiety, an affinity binding moiety and a nucleic acid moiety. Further examples of separating moieties are provided in Examples 1-3 hereinbelow. It will be appreciated that the separating moiety may further comprise a detectable agent. Examples of detectable agent are provided hereinabove.

According to a preferred embodiment, the substrate of the present invention substrate is comprised in a composition which has the following general formula.

X—Y-Z

wherein:

Y comprises a substrate of a neuraminidase, cleavage of X—Y-Z by the neuraminidase forming cleavage products X—Y′ and Y″-Z wherein Y′ is a first cleavage product of Y and Y″ is a second cleavage product of Y;

X comprises a detectable moiety; and

Z comprises a separating moiety capable of binding to a separate phase of a two phase separating system;

wherein X—Y-Z does not form a contiguous portion of a natural substrate of the viral neuraminidase.

Measures should be taken that the detectable moiety does not bind to the separating moiety.

In any of the embodiments described herein, any of the moieties can be directly linked to the peptide by a covalent bond or indirectly via a spacer molecule having coupling functional groups at each end. Examples of such linkers include an alkyl, a glycol, an ether, a polyether, a polynucleotide and a polypeptide molecule.

As mentioned herein above, the compositions of the present invention may be used for detecting a pathogen in a sample. The method comprises:

(a) contacting the sample with a neuraminidase substrate under conditions allowing cleavage of the substrate; and

(b) monitoring cleavage of the substrate, wherein cleavage of the substrate is indicative of the presence of the at least one pathogen in the sample.

A “pathogen” as used herein, refers to any pathogen that comprises neuraminidase on its cell surface. Exemplary pathogens include, but are not limited to influenza A virus, influenza B virus, streptococcus pneumonia, vibrio cholera, salmonella, seed virus, Newcastle disease virus, Mumps virus, La Piedad-Michoacan-Mexico virus, sandai virus, Measles virus Rinderpest virus, Nipah virus, Parainfluenza, Hendra virus and Respiratory Syncytial Virus (RSV). According to a preferred embodiment of this aspect of the present invention, the pathogen is an influenza virus.

Thus, according to one embodiment of this aspect of the present invention, the composition is contacted with the sample being tested for the presence of influenza virus. If the virus is present in the sample, the viral neuraminidase is also present. This enzyme cleaves the substrate and a change in the signal from the signaling moiety (i.e. detectable moiety) can be observed. Such homogenous fluorescent and calorimetric assays are known to those skilled in the art. See, for example: Biochemistry, Allinger, Wang Q. M. et al., “A continuous calorimetric assay for rhinovirus-14 3C protease using peptide p-nitroanilides as substrates” Anal. Biochem. Vol. 252, pp. 238-45 (1997), and Basak S. et al. “In vitro elucidation of substrate specificity and bioassay of proprotein convertase 4 using intramolecularly quenched fluorogenic peptides” Biochem. J. Vol. 380, pp. 505-14 (2004).

As used herein the term “sample” refers to any biological sample (e.g., tissue culture sample or body fluid/tissue sample) which may comprise or permissive for the virus. Preferably the biological sample refers to body fluids such as whole blood, serum, plasma, cerebrospinal fluid, urine, lymph fluids, various external secretions of the respiratory (e.g., nasal wash sample), intestinal, and genitourinary tracts, tears, saliva, semen, sweat, feces, and milk, as well as white blood cells, malignant tissues, amniotic fluid, and chorionic villi.

The biological sample may be derived from any organism, preferably a mammal (e.g., human) or avian.

As mentioned hereinabove, the method of the present invention is effected by contacting a sample with a neuraminidase substrate. Preferably, the neuraminidase substrate comprises the compositions of the present invention, as described hereinabove. However, it will be appreciated that many neuraminidase substrates are known in the art and may be used according to this aspect of the present invention (see e.g. U.S. Pat. No. 7,081,352).

Preferably, the sample is contacted with the substrate of the present invention in the presence of a buffer which includes enzyme inhibitors. Any buffer may be used according to this aspect of the present invention as long as it does not interfere with the cleavage process.

According to one embodiment of this aspect of the present invention, the sample is left in contact with the substrate composition for a sufficient amount of time so that cleavage of all substrate molecules may take place.

According to another embodiment of this aspect of the present invention, the sample is left in contact with the substrate composition for an amount of time so that the initial cleavage kinetics may be monitored.

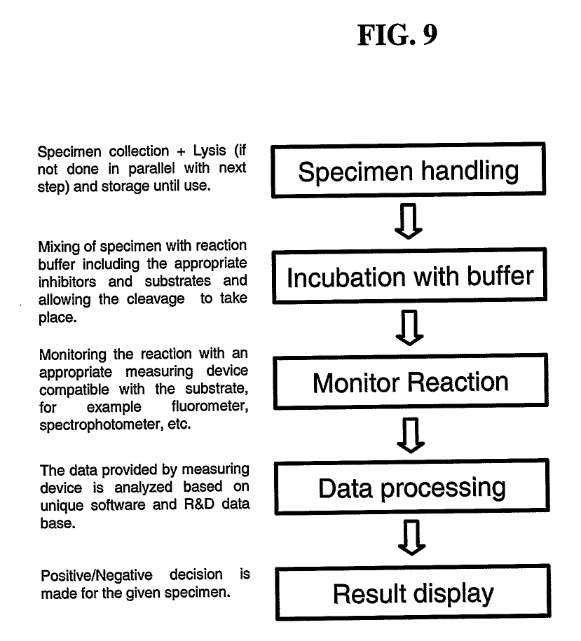

According to yet another embodiment the monitoring is effected throughout the cleavage process (i.e. real time monitoring). FIG. 9 illustrates a particular embodiment of the method of the present invention.

It will be appreciated that the method of the present invention may also be used to differentiate between viral strains. Different strains of a particular virus comprise different affinities for the substrate molecules of the present invention. For example a first influenza virus strain may comprise a neuraminidase that is efficient in cleaving sialic acid when it is presented by an Hi polypeptide whereas a second influenza virus strain may comprise a neuraminidase that is efficient in cleaving sialic acid when it is presented by an H2 polypeptide. Likewise, the link between the sialic acid and the carbohydrate (i.e. 2,3, 2,6 or 2,8 link) also confers specificity. Thus, by contacting a sample with a known substrate, it is possible to classify and/or differentiate between viral strains. Exemplary hemagluttinin and neuraminidase pairs associated with particular viruses are presented in Table 5, Example 9.

The method particularly fits monitoring vaccination of avians and humans, since it may be used to detect if the vaccinated species has not developed a violent type of virus.

The method of the present invention may be adapted so that it may be used to distinguish between a human pathogenic avian virus and a human non-pathogenic virus. For example if an avian sample cleaves a sialic acid substrate comprising an α2,6 linkage, this would be indicative of a potentially human pathogenic avian influenza virus.

The method of the present invention may also be used to determine if a human subject has been infected with an avian influenza virus. For example if a human samples cleaves a sialic acid substrate comprising an NeuGcα2,3 linkage, this would be indicative of an avian influenza virus infection.

Any assay known in the art for monitoring substrate cleavage can be used in accordance with this aspect of the present invention.

Monitoring substrate cleavage can be effected by either a homogenous or a heterogeneous assay.

As used herein the phrase “homogeneous assay” refers to an assay not requiring separation of signaling moiety from other assay components.

Using the detection methods described herein, the test sample is contacted with the substrate under conditions that allow cleavage of the substrate by the neuraminidase if the virus is present in the sample. In one embodiment, the temperature is controlled. For example, the temperature can be controlled at 37° C. to provide optimal conditions for the enzyme reaction. The signal from the cleaved substrate fragment is then detected using a detection device appropriate to the label used.

A “heterogeneous assay” is an assay in which the solid-phase is separated from another assay component during the assay.

Solid-phases suitable for use in the heterogeneous assay include, but are not limited to test tubes, microtiter plates, microtiter wells, beads, dipsticks, polymer microparticles, magnetic microparticles, nitrocellulose, chip arrays and other solid phases familiar to those skilled in the art. The signaling moiety used in the heterogeneous assay may be any label known to those skilled in the art. Such labels include radioactive, calorimetric, fluorescent and luminescent labels.

A heterogeneous chemiluminescent assay for the detection of proteases is described in U.S. Pat. No. 56,243,980, the contents of which are incorporated by reference. In one embodiment, the homogeneous or heterogeneous assay method of the present invention is automated so that a result can be obtained without the need for medical staff to be exposed to a subject thought to be infected by the viral disease under test. For example, the subject can be tested in a clean room (for example, but not limited to P3 type room). The subject can pick up, or get before entering the room, a diagnostic kit, which can include a solid phase coated with a substrae of the type discussed above. For example, the solid phase can be a tissue which was previously immersed with substrate, or a test stick that can be from the type used to test pregnancy. The subject can supply a sample, such as a saliva sample, at a pre-prepared spot on the solid phase.

The solid phase containing the sample is then incubated to allow the enzymatic reaction to occur. In one embodiment, the reaction temperature in controlled at 37° C. to provide optimal conditions for the enzyme reaction. When the incubation is complete, the sample to be tested can be measured on a spectrophotometer, using a remote control, or a mechanical system operated manually from outside the room. The sample can be tested for a qualitative color or UV detection. After the test the sample can be discarded by an automated system, or a remote operated handle that trashes the sample.

By combining on one plate several substrates of the present invention, each one comprising a particular combination of silaic acid, carbohydrate and hemagluttinin and building a profile of cleavage of each matrix for each type of neuraminidase a system may be created capable of predicting the next type of virus. This can be used for predicting the vaccine type for the season, or detecting the exact type of the influenza virus, and its degree of letality and violence for human and animals.

In order to detect the presence of a number of viruses at once, it is possible to use any of the methods known in the art or the above described methods adapted for detection of multiple viruses.

The following provides non-exhaustive examples of such means.

Microplate—in a X well plate. Each well contains a different and specific substrate corresponding to different viruses. With the addition of the clinical sample, the reaction is monitored using a standard microplate reader at the appropriate wavelengths and records which wells demonstrated enzymatic activity. Since each well contains one specific substrate it is possible to elucidate which viral neuraminidase is present in the clinical sample according to the data provided by the microplate reader. The presence of the enzymes confirms the presence of the viruses.

Medisel chip technology (Schiffenbauer et al. 2002 Anticancer Res. 22:2663-9) —using Medisel technology it is possible immobilize the specific substrate molecules (corresponding to the viruses of interest) on a chip. With the addition of the clinical sample the reaction is monitored using a laser beam. Since each point on the chip contains one specific substrate it is possible to elucidate which viral enzymes are present in the clinical sample according to the data provided by Medisel device. The presence of the enzymes confirms the presence of the viruses.

Separation on column—Specific substrates (corresponding to the viruses of interest) can be attached to beads from a commercial source with a unique DNA spacer. With the addition of the clinical sample the reaction is carried out. Once cleavage of a specific substrate occurs (by the specific viral neuraminidase in the clinical sample) the quencher is released and the bead emits fluorescence. The beads are then separated on a column via hybridization to the unique DNA spacer and fluorescent is measured for each type of bead (corresponding to each different virus) using a standard fluorometer at the appropriate wavelengths. Only beads that were cut by the viral neuraminidase emits fluorescence. It is then possible to elucidate which viral neuraminidases are present in the clinical sample. The presence of the enzymes confirms the presence of the viruses.

Bead FACS separation—Similar to column separation only, the separation step is done by FACS when each specific peptide is attached to a bead with different color. In this method the spacer can be either DNA or peptide or peptide-mimetic or carbohydrate or any organic moiety spacer [Gonzalez (2005) Clin. Biochem. 38:966-72].

Other methods which can be used in accordance with the present invention are described by Tozzoli et al. (2006) Clin. Chem. Lab Med. 44:837-42; Abreu (2005) Ann. N.Y. Acad. Sci. 1050:357-63; Buliard (2005) Ann. Biol. Clin. (Paris) 63:51-8; Yinnaki (2004) J. Immunoassay Immunochem. 25:345-57; Rouquette (2003) 120:676-81; Toellner (2006) Clinical Chemistry 52:1575-1583; Horejsh (2005) Nucl. Acids Res. 33, each of which is incorporated herein by reference in its entirety.

Kits which comprise the neuraminidase substrates of the present invention are also envisaged. The different kit components may be packaged in separate containers and admixed immediately before use. Such packaging of the components separately may permit long-term storage without losing the active components' functions. Embodiments in which two or more of components are found in the same container are also contemplated. An exemplary kit may comprise one or more of the following reagents: a wash buffer reagent for use using heterogeneous assays; a negative control reagent free of a neuraminidase capable of cleaving substrate; a positive control containing a neuraminidase capable of cleaving the substrate; (d) a signal generation reagent for development of a detectable signal from the signaling moiety; and (d) a sample collection means such as a syringe, throat swab, or other sample collection device.

The kits of the present invention may, if desired, be presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

The kit of the present invention may further comprise other substrates (e.g. protease substrates) for detection of other respiratory viruses such as Corona Viruses, SARS, HMPV (Human Meta pneumo virus), Adeno virus, RSV (Respiratory Syncytial Virus) and Rhino virus.

The reagents included in the kits can be supplied in containers of any sort such that the life of the different components are preserved and are not adsorbed or altered by the materials of the container. For example, sealed glass ampules may contain lyophilized reagents, or buffers that have been packaged under a neutral, non-reacting gas, such as nitrogen. Ampules may consist of any suitable material, such as glass, organic polymers, such as polycarbonate, polystyrene, etc. ceramic, metal or any other material typically employed to hold similar reagents. Other examples of suitable containers include simple bottles that may be fabricated from similar substances as ampules, and envelopes, that may comprise foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to be mixed. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Exemplary Substrates and Separating Systems of the Present Invention

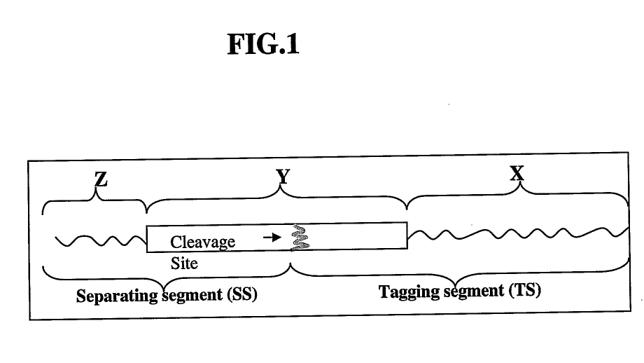

FIG. 1 is a schematic representation of the structure of a substrate of the present invention. The substrate is comprised of 3 parts: X, Y and Z. The core molecule or complex of molecules (segment Y) which has a specific cleavage site is connected in one end to a tagging molecule (X), whose purpose is to detect cleaved substrates. In the other end it is connected to a mechanism segment (Z) that separates between processed and unprocessed substrate. Upon cleavage of molecule Y the substrate is divided into two parts: (1) Tagging Segment (TS) that contains part X and a part of Y and (2) a Separating segment (SS) that contain Z and a part of Y.

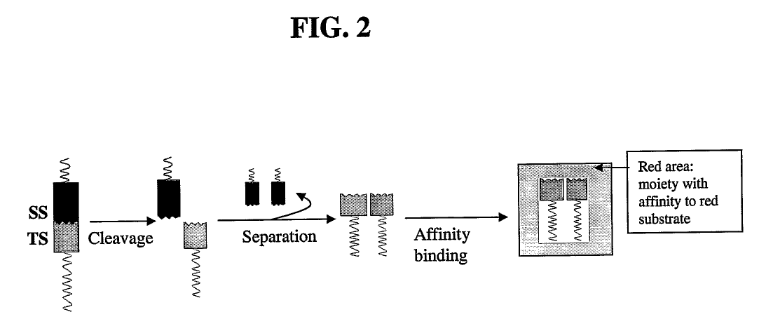

Upon cleavage of the substrate described in FIG. 1 by neuraminidase, the Z segment (separating segment) is used to separate between the processed and unprocessed substrate. Therefore, only the TS of the processed substrate (that contains the tagging molecule) binds to the moiety with the high affinity to the tagging molecule. The affinity binding process is therefore detected only for cleaved substrates. In this way it is possible to detect only processed molecules (FIG. 2).

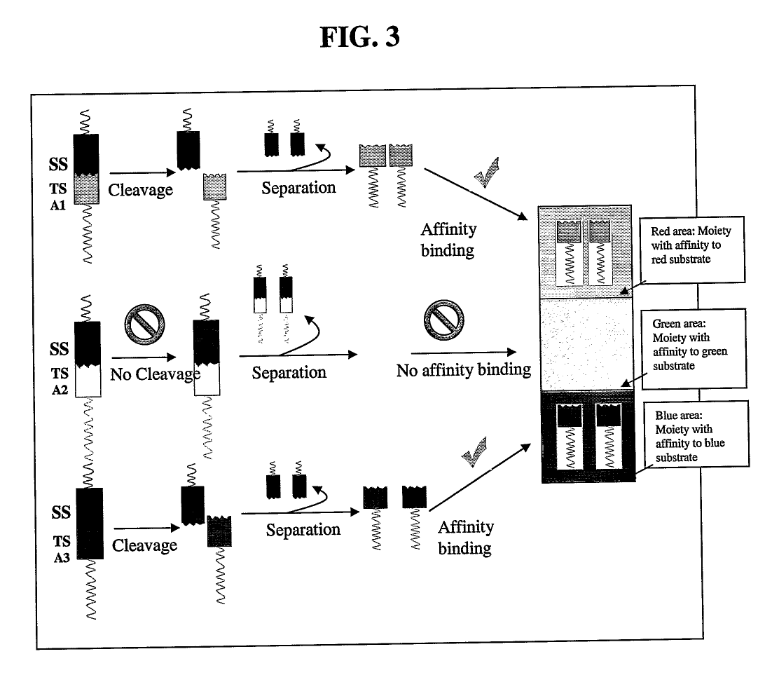

FIG. 3 illustrates the simultaneous detection of cleavage of a number of substrates using the above described method. Simultaneous detection may occur if the substrates are similar in their separating mechanism (Z) but different in their specific cleavage molecule (Y). In this case each substrate has a unique and different tagging molecule (X) that can be affiliated to its core molecule (Y). After cleavage and separating between processed and unprocessed substrates occurs, only the TS of the different (and processed) substrates are bound by affinity. Any molecule that contains Z (unprocessed substrates or SS of processed substrates) is retained by the separating mechanism. Further separation to the different TSs of the different substrates may be obtained by designing a membrane, chip etc. on which each predetermined locus contain a moiety with affinity to each different TS, e.g. each locus can bind only to one kind of TS. The separated solution then comes in contact with this chip or membrane and affinity binding can occur. By knowing which predetermined loci are bound by affinity to the different TSs one can identify which substrates were processed. Since each substrate is specific to the enzyme that initiated the substrate cleavage one can identify which of the enzymes have cleaved its corresponding substrate, deduce which neuraminidase exists in the solution and consequently deduce which pathogen or agent corresponds to the corresponding enzyme.

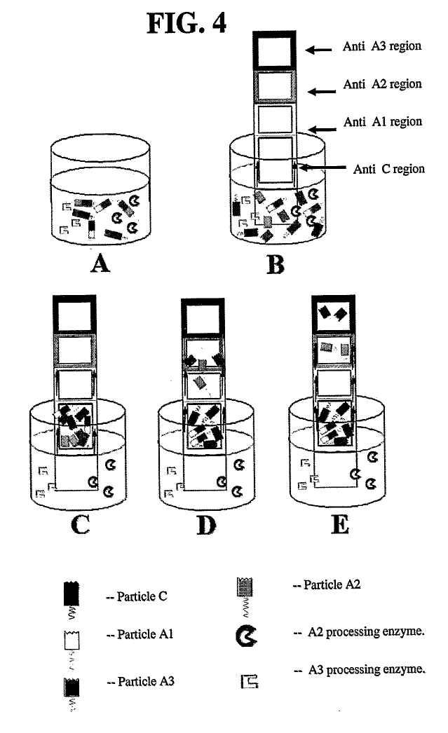

Exemplary Separating Systems:
1. Immobilized Separation System (ISS)—in this system Z is a spacer linked to an immobilized surface via beads, nitrocellulose membrane, biotin-avidin or other affinity pair. After cleavage in a buffered solution any unprocessed substrate or the SS of the processed substrate is removed by separating the immobilized surface (by extraction, centrifugation, filtration etc.) from the buffered solution, leaving only the TS of the processed substrates. In this way it is also possible to monitor the kinetics of each substrate.
2. Dynamic Separation System (DSS)—in this system Z is a special molecule common or unique to all substrates in the buffered solution. After cleavage occurs, the buffered solution comes in contact with a specially designed membrane or chip. The membrane is vertical and at the bottom it has a moiety with affinity corresponding to Z. An adjacent part of the membrane includes different loci corresponding to X of the different substrates. The buffered solution is then pushed along the length of the membrane or chip by capillary or electro force. Any molecule that contains Z (unprocessed substrates or SS of processed substrates) will be retained at the bottom of the membrane (by the affinity moiety corresponding to Z). Only the TS of the processed substrates (do not contain Z) will be able to move up the membrane and bind by affinity to their predetermined loci (FIG. 4).
3. Affinity Filtration System (AFS) in this system the buffered solution is filtered through a column with affinity to Z, thus any molecule that contains Z (unprocessed substrates or SS of processed substrates)—will remain in the column. The flow trough will contain only the TS of the processed substrates.

### Example 2

### Neuraminidase Detection Based on Beads and Fluorescence (FRET)

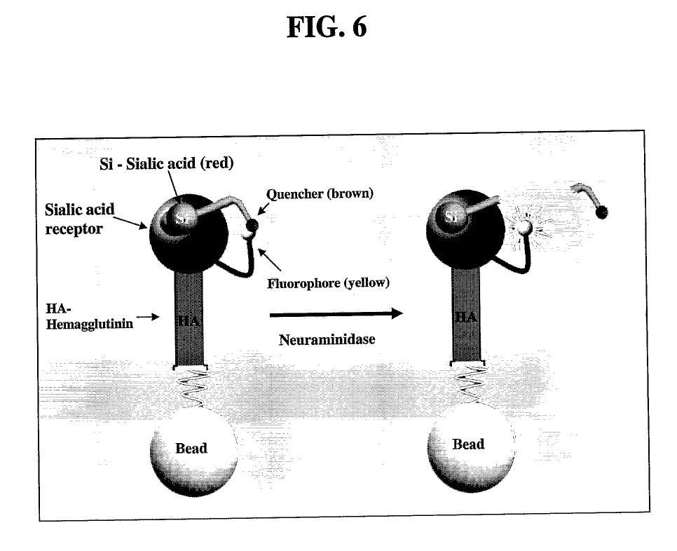

This example describes the detection of a presence of avian flu H5N1 virus and other types of influenza in a clinical sample. The influenza family of viruses utilize combinations of specific hemagglutinin and neuraminidase pairs. These pairs exhibit specific cleaving activity. In this example the substrate consists of Hemagglutinin (HA) (or parts of it) with a fluorophore connected to a colored bead (corresponding to Z) on one end. A fluorophore may also be attached either to the HA moiety The HA sialic acid receptor is used to bind between the HA moiety one end and the molecule containing the sialic acid and quencher on the other end. Exemplary sialic acid moieties are illustrated in FIG. 5. The other end of the HA molecule ends with the sialic acid receptor which is connected to a sialic acid (corresponding to Y) with a quencher as its residue molecule (corresponding to X) via receptor-substrate affinity (mimicking a natural substrate of neuraminidase). Cleavage of this substrate will result in the separation of the quencher and fluorescence of the bead (FIG. 6).

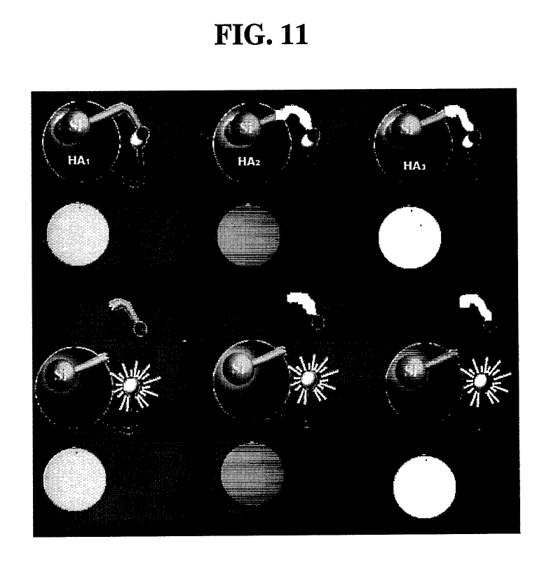

The reaction mixture contains different types of the substrate described above. Each substrate has a different bead color and different linkage to the sialic acid e.g. 2,3 or 2,6 (See FIG. 11). The specificity will be defined according to the complex that is created upon cleavage between the neuraminidase, hemagglutinin, and the sialic acid. Due to the different specificity to the different complexes, substrates will be cleaved at different rates. In such a case, each bead can be affiliated to a different NA of the influenza virus family. The reaction mixture comes in contact with the clinical sample and cleavage of the sialic acid can occur by neuraminidase, removing the quencher part of the molecule. After the cleavage occurs, the beads are separated according to different colors using FACS or other existing technologies and fluorescence (at the appropriate wavelength) is measured for each bead type. Since each bead represents a different neuraminidase cleavage site, it is possible to identify which subfamily of the influenza viruses are present in the clinical sample.

It is also possible to abolish the need for HA by designing a substrate with a spacer instead of HA. The sialic acid is covalently attached to the spacer, via one of its residues in such way that it will not interfere with the catalytic activity of the NA (attached to position 3 or 4 on neuraminic acid)—(covalent bond instead of receptor-substrate affinity bond).

It is also possible to replace the beads with any of the other affinity pairs for separation purposes. Thus, allowing neuraminidase detection by any of the other methods described in the examples. FRET pairs can also consists of quantum dots instead of using beads for detecting cleavage (http://www.azonano.com/Details.asp?ArticleID=1726#_Quantum_Dot_Technology).

### Example 3

### Matrix Determination of Influenza Types (Profiling) for Detection of Unknown Type of Influenza Virus

Influenza viruses are defined according to the combination of Neuraminidase and HA viral proteins. The name of the virus is defined according to this combination. i.e., H5N1 virus has a combination of HA 5 and neuraminidase 1 on its spike. Due to the multiple mutations and evolution in the virus it develops different subtypes every season. Since there is a global fear that one mutated type becomes a specially violent one (as occurred during the Spanish flu period that killed more then 25 million people in Europe in 1918) there is an urgent need to rapidly detect and isolate the violent types of influenza virus, in order to monitor and minimize the spread of the pandemic threatened to occur by such viruses. Currently there is no way to determine in a rapid test the exact profile of a specific influenza type in a certain flu season. The method described in this invention enables such a determination.

In the following example a baseline matrix of substrates is constructed from a combination of H1-Hn proteins attached to several types of sialic acid derivatives linked via 2-3 and 2-6 linkage and comprising different substrate specificities. The panel of substrates is reacted with a panel of neuraminidase enzymes to establish specific profiles of fluorescence. The next step is applying the panel of substrates on an unknown clinical sample. An example of the results obtained is shown in Table 1 hereinbelow. These results are compared to the predetermined profiles in order to determine the specific type of virus present in the clinical sample.

TABLE 4

| NeuGcα2- | | + | | | | |
|---|---|---|---|---|---|---|
| 6Gal-R | | | | | | |
| NeuAcα 2- | | | | | | |
| 3Gal-R | | | | | | |
| NeuAcα 2- | | + | + | + | + | |
| 6Gal-R | | | | | | |
| Substrate | H | H | H | H | H | . . . |
| | 1 | 2 | 3 | 4 | 5 | . . . |

| | |
|---|---|
| NA strain | . . . |

### Example 4

### Neuraminidase Detection Based on Compartmentalization

In this example the reaction is separated to different compartments. Each compartment contains a different substrate with different linkage to the sialic acid, (i.e., Neu5ac2-6gal, neu5ac2-3gal Neu-gly-2-3gal) representing specificity to different neuraminidase. The substrate is a sialic acid (correspond to Y) and a biotin as its residue (corresponds to Z).

Following cleavage each compartment is filtered through a filter with avidin. Any molecule that contains biotin (unprocessed substrate or biotin residue of processed substrate) is retained at the filter. The sialic acid of processed substrate is allowed to pass the filter to a segment of the compartment that contains an assay for sialic acid detection. The assay can be based on thio-barbituric acid on one hand. On another hand ketone derivative of sialic acid as substrate can be used, and thus ketone derivatization detection assay is possible as well. In this method it is determined which compartments undergo cleavage. Since each compartment represents a different neuraminidase cleavage site, it is possible to know which subfamily of the influenza viruses are present in the clinical sample.

### Example 5

### Protease and Neuraminidase Simultaneous Detection Based on Marker (ISS)

In this example the separation step is already achieved by connecting the substrates to an immobilized surface. The substrates for neuraminidase is as described in Example 2 only instead of beads the HA is connected to a predetermined loci on a membrane, chip etc, specially designed for this purpose. The substrate for proteases is a cleavage sequence (corresponding to Y) connected at one end to a predetermined loci on the membrane, chip etc, via a spacer, if one is required (corresponding to Z). X is based on FRET technology in such way that cleavage of the protease removes a quencher from the substrate. Cleavage of any of the substrates will result in fluorescent intensity.

The membrane then comes in contact with the clinical sample and cleavage can occur. By measuring fluorescent intensity for each locus individually processed substrates may be detected. Since each locus represents a different neuraminidase or protease cleavage site, it is possible to know which viruses are present in the clinical sample.

### Example 6

### Human Pathogenic and Non-Pathogenic Avian Influenza Veterinarian Detection Kit

The following example describes an exemplary avian influenza virus detection kit.

N-acetylneuraminic acid (NeuAc) bound to galactose through an α2,3 linkage (NeuAcα 2-3Gal) is preferentially expressed on duck epithelial cells. Human airway epithelium surface exhibits NeuAcα 2-3Gal and NeuAcα 2-6Gal glycoproteins. Avian influenza viruses that are potentially pathogenic to humans should process both NeuAcα 2-3Gal and NeuAcα 2-6Gal based substrates, while avian influenza viruses that are not pathogenic to humans should process only NeuAcα 2-3Gal based substrate.

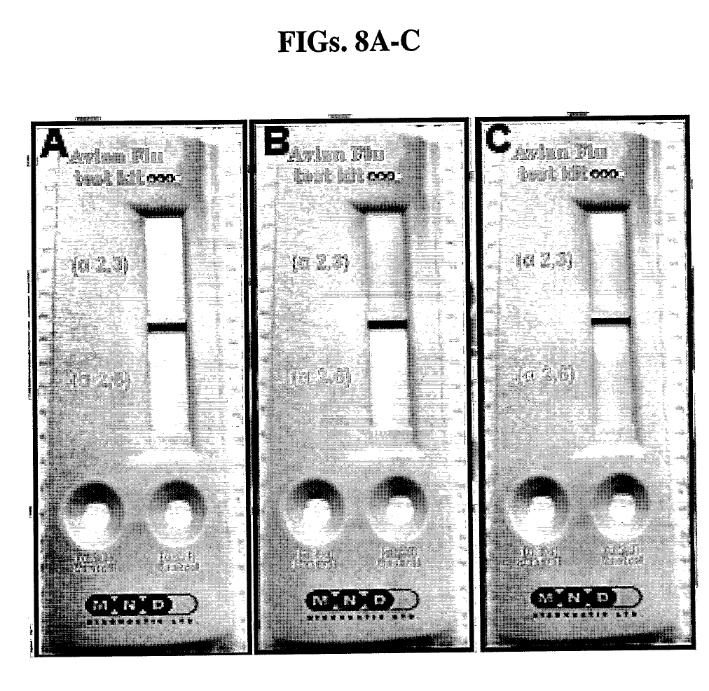

The present example envisages a kit that contains both substrates attached to a solid surface and all the reagents needed to perform the test. A clinical specimen is extracted from a diseased bird. The specimen then comes in contact with solid surface and cleavage can occur. If no substrate is cut (FIG. 8A) the disease is not influenza. If only the NeuAcα 2-3Gal based substrate is cut (FIG. 8B) then the disease is an avian influenza virus which is not pathogenic to humans. If both NeuAcα 2-3Gal and NeuAcα 2-6Gal based substrates are cut (FIG. 8C) then the disease is a human pathogenic avian influenza virus.

### Example 7

### Human Influenza and Avian Influenza Detection Kit in Humans

The following example describes an exemplary kit for detecting if a virus is a human influenza or a human pathogenic avian influenza.

Human tracheal epithelial cells do not possess detectable levels of N-glycolylneuraminic (NeuGcα2-3Gal) acid current human influenza strains exhibit low NeuGc specificity. Bird epithelial cells do possess detectable levels of NeuGcα2-3Gal.

The present example envisages a kit that contains the above described substrate and NeuAcα 2-3Gal both of which are attached to a solid surface. The kit also contains all the reagents needed to perform the test.

A clinical specimen is extracted from a human patient. The specimen is contacted with the solid surface so that cleavage may occur. If no substrate is cut the disease is not influenza (FIG. 8A). If only the NeuAcα 2-3Gal based substrate is cut (FIG. 8B) then the disease is human influenza virus. If both NeuAcα 2-3Gal and NeuGcα 2-3Gal based substrates are cut (FIG. 8C) then the disease is human pathogenic avian influenza virus.

### Example 8

### Detection Using a Pre-Substrate

The pre-substrate design is as follows: Neu5Ac is connected via an α2,3 to the C3 of the galactose of Xgal. Xgal is a known chromogenic substrate of β-galactosidase (FIG. 10).

The pre-substrate comes in contact with the clinical specimen containing influenza. The NA in the specimen cleaves the pre-substrate. Cleavage of the pre-substrate generates free sialic acid and free Xgal. The β-galactosidase already present in the media can then process the Xgal and catalyze the chromogenic reaction. The medium color turns blue.

If the clinical specimen does not contain influenza the sialic acid remains bound to the Xgal. The β-galactosidase (which, can process only terminal galactose containing substrates) can not process the Xgal and the chromogenic reaction will not occur. The medium color does not change color.

### Example 9

### Hemagluttinin and Neuraminidase Pairs

The following example tabulates exemplary influenza virus hemagluttinins and neuraminidase pairs which may be used in the diagnosis of influenza viruses.

Lengthy table referenced here
US20090220941A1-20090903-T00001
Please refer to the end of the specification for access instructions.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

LENGTHY TABLES
The patent application contains a lengthy table section. A copy of the table is available in electronic form from the USPTO web site (http://seqdata.uspto.gov/?pageRequest=docDetail&DocID=US20090220941A1). An electronic copy of the table will also be available from the USPTO upon request and payment of the fee set forth in 37 CFR 1.19(b)(3).

1.-62. (canceled)

63. An isolated composition-of-matter comprising a sialic acid bound to a sialic acid binding domain of a polypeptide.

64. The isolated composition-of-matter of claim 63, wherein said sialic acid is attached to at least one carbohydrate via a linker.

65. The isolated composition-of-matter of claim 64, wherein said sialic acid is attached to two carbohydrates wherein a first carbohydrate of said two carbohydrates is attached to a second carbohydrate of said two carbohydrates via a second linker.

66. The isolated composition-of-matter of claim 65, wherein said second linker is selected from the group consisting of an α1-4 linker, an α 1-3 linker, an cc2-3 linker and an α 1-6 linker.

67. The isolated composition-of-matter of claim 63, being hydrolizable by neuraminidase.

68. The isolated composition-of-matter of claim 64, wherein said linker is selected from the group consisting of an α2,3 linker, an α2,6 linker and an α2,8 linker.

69. The isolated composition-of-matter of claim 63, wherein said polypeptide is a hemagglutinin polypeptide.

70. The isolated composition-of-matter of claim 63, further comprising at least one detectable moiety.

71. The isolated composition-of-matter of claim 63, further comprising a separating moiety.

72. A composition being of the general formula: X—Y-Z wherein:
Y comprises a substrate of a neuraminidase, cleavage of X—Y-Z by said neuraminidase forming cleavage products X—Y′ and Y″-Z wherein Y′ is a first cleavage product of Y and Y″ is a second cleavage product of Y;
X comprises a detectable moiety; and
Z comprises a separating moiety capable of binding to a separate phase of a two phase separating system; wherein said X—Y-Z does not form. a contiguous portion of a natural substrate of said viral neuraminidase.

73. The composition of claim 72, wherein said separating moiety further comprises a detectable moiety.

74. A method for detecting at least one pathogen in a sample, the method comprising:
(a) contacting the sample with the compositions of claim 1 under conditions allowing cleavage of said substrate; and
(b) monitoring cleavage of said substrate, wherein said cleavage of said substrate is indicative of the presence of said at least one pathogen in the sample.

75. The method of claim 74, wherein the pathogen is an influenza virus.

76. The method of claim 74, wherein when said substrate comprises an α2,6 linkage, said cleavage of said substrate is indicative of a potentially human pathogenic avian influenza virus.

77. The method of claim 74, wherein when said substrate comprises an NeuGcα2,3 linkage, said cleavage of said substrate is indicative of avian influenza virus.

78. A method of detecting a pathogenic avian influenza virus in a sample, the method comprising
(a) contacting a sample with a neuraminidase substrate; and
(b) monitoring cleavage of said substrate, wherein said cleavage of said substrate is indicative of a pathogenic avian influenza virus.

79. A method according to claim 78, wherein said neuraminidase substrate comprises a linkage selected from the group consisting of a NeuGcα2,3 linkage and an α2,6 linkage.

80. The method of claim 78 wherein said sample is selected from the group consisting of mucus, saliva, throat wash, nasal wash, spinal fluid, sputum, urine, semen, sweat, feces, plasma, blood, broncheoalveolar fluid, vaginal fluid, tear fluid and tissue biopsy.

81. The method of any of claim 78, wherein said monitoring is effected using an assay selected from the group consisting of a homogeneous assay and a heterogeneous assay.

82. A diagnostic kit comprising a packaging material and a plurality of compositions for detecting presence of a plurality of influenza viruses in a sample, wherein each of said compositions comprises a composition according to claim 63.

83. A diagnostic kit comprising a packaging material and a plurality of compositions for detecting presence of a plurality of influenza viruses in a sample, wherein each of said compositions comprises a composition according to claim 72 and, wherein each of said X is distinctively detectable and whereas said packaging material comprises a label or package insert indicating that the kit is for detection of plurality of influenza viruses in a sample.

84. The diagnostic kit of claim 83, wherein said substrate of said viral neuraminidase comprises a sialic acid bound to a sialic acid binding domain of a polypeptide.

85. The diagnostic kit of claim 83, further comprising reagents for detecting cleavage of said substrate.

86. The diagnostic kit of claim 83, wherein each of said plurality of compositions is a substrate for a different neuraminidase.

87. The diagnostic kit of claim 83, wherein said plurality of compositions are attached to a single solid support.

88. The diagnostic kit of claim 87, wherein said distinctive detection is effected by an addressable location on said single solid support.

89. The diagnostic kit of claim 83, wherein said distinctive detection is effected by different detectable moieties.
